(19) United States
(12) Patent Application Publication
(10) Pub. No.: US 2006/0073471 A1
(43) Pub. Date: Apr. 6, 2006

(54) Method of using molecularly imprinted polymers for noninvasive diagnosis

(76) Inventors: Joseph J. Bango, New Haven, CT (US); Michael E. Dziekan, Bethany, CT (US)

Correspondence Address: CONNECTICUT ANALYTICAL CORPORATION;JOSEPH J. BANGO, 696 AMITY ROAD, BETHANY, CT, 06524, US

(21) Appl. No.: 11/244,094

(22) Filed: Oct. 5, 2005

Related U.S. Application Data
(60) Provisional application No. 60/616,503, filed on Oct. 5, 2004.

(51) Int. Cl. C12Q 1/00; G06G 7/48; G06G 7/58

(52) U.S. Cl. 435/4; 703/11

(57) ABSTRACT

The invention outlines a method for using Molecularly Imprinted Polymers (MIP's) in conjunction with specific, narrow wavelength light sources to non-invasively test for disease precursors. The invention makes it possible for a real time analysis to be carried out on the human body with minimal effort and greater efficiency than traditional methods.

### CROSS REFERENCE TO RELATED APPLICATIONS

Provisional Application No. 60/616503 was filed on 5 Oct. 2004

### BACKGROUND—FIELD OF INVENTION

The invention outlines a method for using Molecularly Imprinted Polymers (MIP's) in conjunction with specific, narrow wavelength light sources to non-invasively test for disease precursors. The invention makes it possible for a real time analysis to be carried out on the human body with minimal effort and greater efficiency.

### BACKGROUND DESCRIPTION OF PRIOR ART

Current medical research has found that some animals, dogs in particular, have an unusual ability to smell certain diseases in humans. There have been numerous reports of dogs that sniff moles on a person's skin for the purposes of detecting melanoma, or cancerous skin cells. The same has been applied to having specially trained dogs detect for the presence of Pancreatic cancer in urine samples taken from a patient. Others have been researching how a persons breath can convey information as to a person's health. The described invention details how to utilize a persons breath, and urine to test for various ailments and cancers. The described invention allows for a totally non-invasive laboratory test to be performed, without the need for drawing blood, or performing a biopsy.

One preferred method of chemical identification is through the use of specially designed MIP's or Molecularly Imprinted Polymers. MIP's have been used for some time in various testing methodologies. The structure of a MIP can best be described as a synthetic antigen that has various bonding sites that selectively target very specific molecules. The MIP has an advantage in that it is very robust and can be used over and over without degradation. MIP's have been used to test for the presence of dangerous chemicals, and compounds indicative of biological agents. The MIP can be designed with characteristics that will enable the targeting of desired molecules with a high degree of specificity. Several MIP families can be present on the same surface set in a striped or checkerboard pattern that will enable a matrix of MIP's to generate a characteristic fingerprint for various compounds. If a known database of “fingerprints” is compared to a test compound, then a quick determination can be made to help identify the unknown substance.

### BRIEF DESCRIPTION OF THE DRAWINGS

FIG. 1 shows a small, disposable plastic bag that will be used to test for the presence of various compounds contained in human or animal breath. The plastic bag is designed to capture a known volume of air from the lungs and help concentrate the gaseous compounds onto the MIP's. The bag is designed with a special valve that lets air in, but prevents it from escaping.

FIG. 2 shows one of the MIP laced plastic bags in the unused, empty form, in the process of being filled, and fully filled. The fully filled plastic bag will then be tested for the presence of various compounds.

FIG. 3 shows a light-proof testing chamber that will be used to test for the presence of specific compounds contained in the sample inside the plastic bag. The MIP's will target and concentrate specific molecules to test for the presence of various diseases and ailments. High intensity, specific wavelength sources are used in conjunction with a suitable light sensing device such as a photomultiplier tube, or a CCD (Charge Coupled Device) camera connected to a PC (Personal Computer).

FIG. 4 shows a top view of one of the plastic bags for the purposes of indicating various discrete regions of specific MIP's. Some are used only as a test control to indicate operational functionality and for reference. Each discrete region could have a MIP designed for a different molecule; the combination of several regions shown as active will help to determine specific fingerprints of different compounds. As each discrete region glows under the influence of suitable wavelength light (fluoresces), a determination can be made as to the type and concentration of each molecule can be made.

FIG. 5 shows several views of a tray that could be used to contain a discrete amount of liquid for testing. The liquid could be urine, and with the help of specially designed MIP's coated onto the base of the tray, a determination can be made as to the molecules contained in the urine.

FIG. 6 shows a top and side view of one of the disposable liquid sample trays for the purposes of indicating various discrete regions of specific MIP's. Some are used only as a test control to indicate operational functionality and for reference. Each discrete region could have a MIP designed for a different molecule; the combination of several regions shown as active will help to determine specific fingerprints of different compounds. As each discrete region glows under the influence of suitable wavelength light (fluoresces), a determination can be made as to the type and concentration of each molecule can be made.

FIG. 7 shows a light-proof testing chamber that will be used to test for the presence of specific compounds contained in the sample inside the disposable liquid sample tray. The MIP's will target and concentrate specific molecules to test for the presence of various diseases and ailments. High intensity, specific wavelength sources are used in conjunction with a suitable light sensing device such as a photomultiplier tube, or a CCD (Charge Coupled Device) camera connected to a PC (Personal Computer).

FIG. 8 shows a top view of one of the disposable liquid sample trays for the purposes of indicating various discrete regions of specific MIP's. Some are used only as a test control to indicate operational functionality and for reference. Each discrete region could have a MIP designed for a different molecule; the combination of several regions shown as active will help to determine specific fingerprints of different compounds. As each discrete region glows under the influence of suitable wavelength light (fluoresces), a determination can be made as to the type and concentration of each molecule can be made.

### DETAILED DESCRIPTION OF THE INVENTION

In the field of medical research, it has been found that some animals, dogs in particular, have an unusual ability to smell certain diseases, or components such as specific biomarkers in humans. Numerous reports have been noted of dogs that sniff moles on a person's skin to detect melanoma, or cancerous skin cells. Similar research has been applied to examining human urine samples for various diseases and ailments. By having specially trained dogs smell the urine sample to detect for the presence of Pancreatic cancer or other ailments. Others have been researching how a persons breath can convey information as to a person's health. The described invention details how to non-invasively analyze a persons breath and/or urine to test for various ailments and cancers without the need for drawing blood or taking a biopsy.

Throughout the years, many people have tried to create a synthetic nose that is as sensitive as a dog's nose. Almost everybody has at one time or another witnessed how a few Bloodhounds can pick up the scent of an escaped convict, or a missing child many hours after they have left the area. The ability for a dog to smell the scent of a specific odor from a specific object is unmatched by any human being. Some laboratory instruments are capable of performing similar tests, but only for a very selective range of chemicals, or odors. It would be very cumbersome to drag a large piece of computerized equipment through the woods trying to locate a weak, specific scent buried in a myriad of stronger, unwanted scents. One preferred method of chemical identification is through the use of specially designed MIP's or Molecularly Imprinted Polymers. MIP's have been used for some time in various testing methodologies. The structure of a MIP can best be described as a synthetic antigen that has various bonding sites that selectively target very specific molecules. The MIP has an advantage in that it is very robust and can be used over and over without degradation. MIP's have been used to test for the presence of dangerous chemicals, and compounds indicative of biological agents. A MIP can be designed with characteristics that will enable the targeting of specific molecules with a high degree of specificity and concentrate them to a level where they can be easily detected by optical means, such as fluorescence. A multitude of different MIP families can be present on the same surface set in discrete rows or columns, or in checkerboard pattern that will enable a matrix of MIP's to generate a characteristic fingerprint for various compounds. When a known database of characteristic “fingerprints” is compared to an unknown test compound, a quick determination can be made to help identify the unknown substance.

There are two main modes of operation when using the MIP's to detect for the presence of specific chemical species or biomarkers—fluorescence “extinction” and fluorescence “emission”. When the MIP is operating in the extinction mode, it is composed of material that will normally glow (fluoresce) under the illumination from a specific wavelength source. When the MIP is sequestering its targeted non-fluorescing chemical specie or non-fluorescing biomarker, the MIP will be blocked from fluorescing, and will appear dark or weakly fluorescing. This will give an indication to the user that the specific “extinction mode” MIP's are sequestering their target specie. In the second mode of operation, or fluorescence “emission” mode, the MIP is composed of material that does not fluoresce under the specific wavelength light used to illuminate the sample. The target specie that the MIP is designed to sequester does however fluoresce under the same specific wavelength illumination. When the user notices that specific regions of MIP's that are non-fluorescing begin to fluoresce, a determination can be made that the MIP's are sequestering their target specie or biomarker. A broad spectrum of suitable illumination can be used to illuminate a test sample having combinations of “extinction” and “emission” MIP's. It should be noted that in the preferred embodiment of the described invention, the suitable wavelength illumination source would be swept through a range of various discrete or narrow wavelengths. While sweeping through each individual wavelength, it would be known what wavelength is currently illuminating the MIP's, and the response of all the MIP's to the specific illumination would be recorded. After a small time interval, the next discrete wavelength or very narrow band of wavelengths will illuminate the MIP's. Although it is stated that a “narrow or very narrow band of wavelengths” will be used for illumination, it is preferred that a plurality of single wavelength sources or monochromatic sources are used. The rationale for using a “narrow band of wavelengths” is only stated for allowing the realization of a cheaper illumination source to be used for commercial use. The preferred embodiment will use a broad-spectrum illumination source with the ability to select single or very narrow ranges of wavelengths at a time, similar to using a grating with a laser to tune specific wavelengths. The preferred embodiment if the described invention will also use combinations of MIP's operating in both “extinction” mode and “emission” mode, although they will not be mixed together, but kept in homogenous, discrete, well defined regions, such as rows, columns, or in a checkerboard matrix. Each discrete row, column, or checkerboard square will contain a homogenous grouping of MIP's of the same mode of operation. A single row, column or checkerboard square will not contain a mixture of different mode MIP's, or MIP's that target different species. All the MIP's in a discrete row, column, or square will be of the same mode of operation, and target the identical species. The terminology of row, column, or square is not intended to express a specific geometric shape, but only to convey that a discrete, discernable area is indicated. It could just as easily be a circle, triangle or shape such as that of an alphabet letter or number. The preferred embodiment of the described invention will utilize discrete rows, columns or checkerboard squares, each containing a homogenous grouping of MIP's.

It has been shown that when a person inhales specific biological weapons of mass destruction (WMD's) such as anthrax, the anthrax spore will chemically breakdown or Lyse inside the lung. This gives rise to chemical constituents that could be detected by the described invention. This detection of inhaled biological warfare agents would serve as a means to counter terrorist activities, due to the fact that a person would be diagnosed much earlier than waiting for the person to show the typical signs or symptoms related to anthrax exposure. The difference in time between waiting for a person to show symptoms, and a positive response from the described invention will mean hours or even days. This will equate to more lives saved, in addition to alerting the proper authorities to quarantine a specific area to prevent further exposure. It is in this capacity that the described invention would function as a fast and effective anti-terrorism weapon.

In addition to having a MIP coated liquid sample tray for sampling liquids, a strip of paper or plastic that is coated with MIP's specific to various target species, such as acetone, ammonia, ethyl alcohol, and dipicolinic acid, to name just a few examples, could be used as an alternative to Litmus paper. The MIP's that are coated on the small strip will react and sequester the targeted chemical compounds and molecular species when dipped into a sample of liquid. The strip coated with MIP's could be coated with a homogenous coating, or covered with a plurality of discrete rows, columns, or checkerboard squares of MIP's—each selective or specific to a definite type of molecular specie. The MIP coated strip would be tested in the same test chamber that both the air bag and liquid sample tray are tested in.

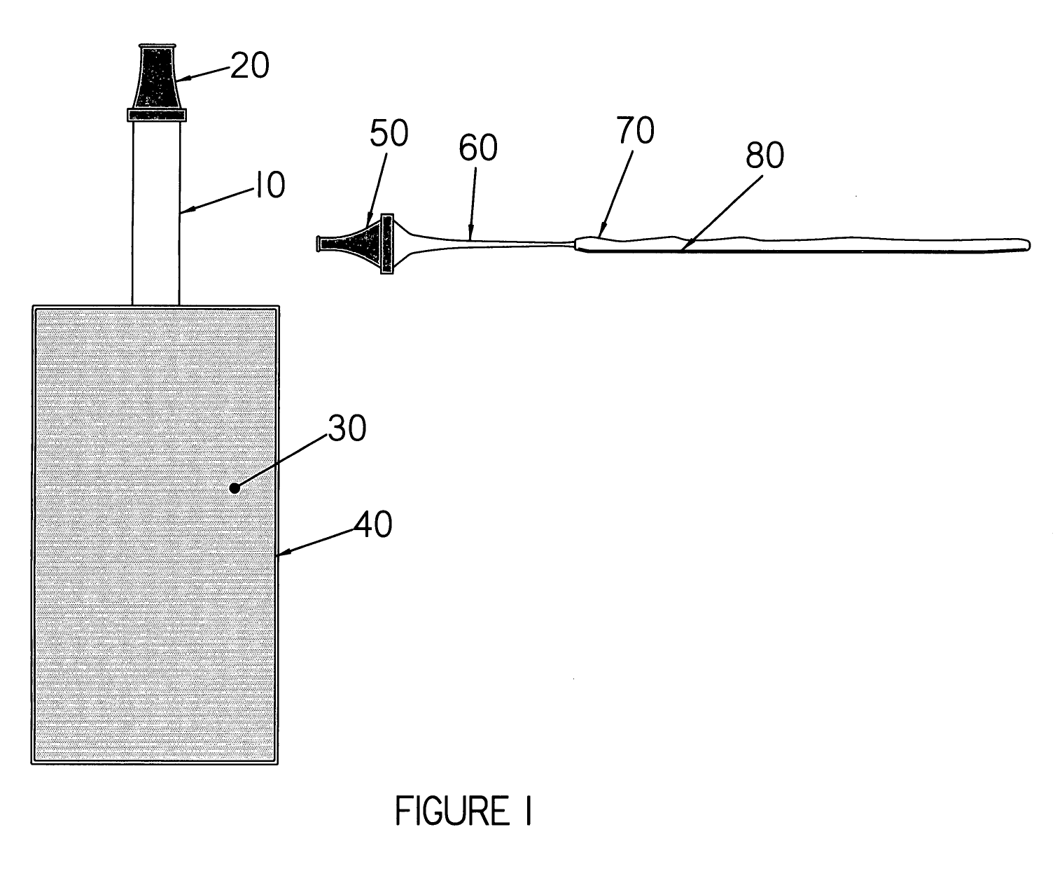

FIG. 1 shows a 2-dimensional top view of an uninflated, flexible plastic disposable air sample bag 40. To inflate the inflatable air sample bag, one needs to blow air into it. This is accomplished by placing ones lips onto the rigid, plastic nozzle 20 that is designed in such a way as to allow air into the flexible air sample bag, but prevent it from escaping. The rigid, plastic nozzle 20 connects to the main body of the flexible air sample bag 40 by a small flexible plastic tube 10 which will channel all the air inside the flexible air sample bag 40. The inside of the flexible air sample bag 40 is coated with a uniform coating of MIP's 30 that are designed to target specific molecular species. A side view of the uninflated bag is shown as 70. When uninflated, the flexible plastic air sample bag 70 is virtually flat, except for the rigid, plastic nozzle 20 that connects to the flexible plastic tube 60 to allow air to enter the air sample bag 70. The inside of the flexible air sample bag is shown in its side view coated with a uniform layer of MIP's 80 that will be used to help identify the chemical components of the air sample. The flexible air sample bag is designed in such a way as to allow the full volume of a persons lungs to be exhaled into the air sample bag for analysis. Several size air sample bags would be used for different patients, from small children, to large adults. Other bags could be used for performing routine air samples from different localities of from emissions from industrial plants. The difference would be in the type of MIP's that are coated inside the air sample bag, and if used to gather air samples from field sources, or near car exhausts, or industrial emissions, a small pump would be needed to inflate the flexible air sample bag to obtain a sample. By using the air sample bag, a repeatable, known quantity of air would always be taken for more accurate and precise testing.

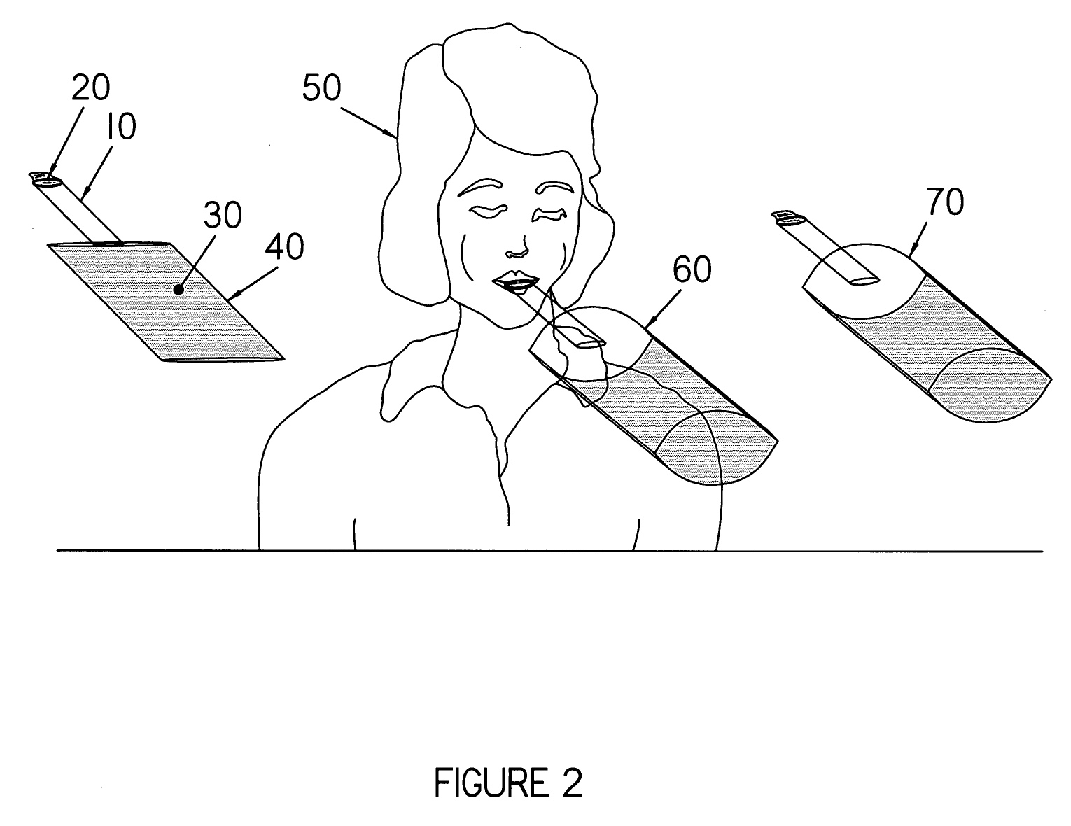

FIG. 2 shows a “real world” application of breath analysis testing. A 3-dimensional view of an uninflated flexible air sample bag is shown 10. In order to inflate the air sample bag, one needs to blow air into it by placing ones lips onto the rigid, plastic nozzle 20 that is designed in such a way as to allow air into the flexible air sample bag, but prevent it from escaping. The rigid, plastic nozzle 20 connects to the main body of the flexible air sample bag 40 by a small flexible plastic tube 10 which will channel all the air inside the flexible air sample bag 40. The inside of the flexible air sample bag 40 is coated with a uniform coating of MIP's 30 that are designed to target specific molecular species. As a person/patient 50 begins the breath analysis testing, they are to exhale a full lung capacity into the flexible air sample bag in one breath. As the person/patient 50 exhales into the flexible air sample bag, it inflates 60 and becomes semi-rigid. When fully inflated, the flexible air bag will contain a full volume of exhaled air 70, and due to the nature of the rigid plastic one way air valve, no air escapes out of the bag and it remains semi-rigid. The fully inflated air sample bag 70 is now ready for testing.

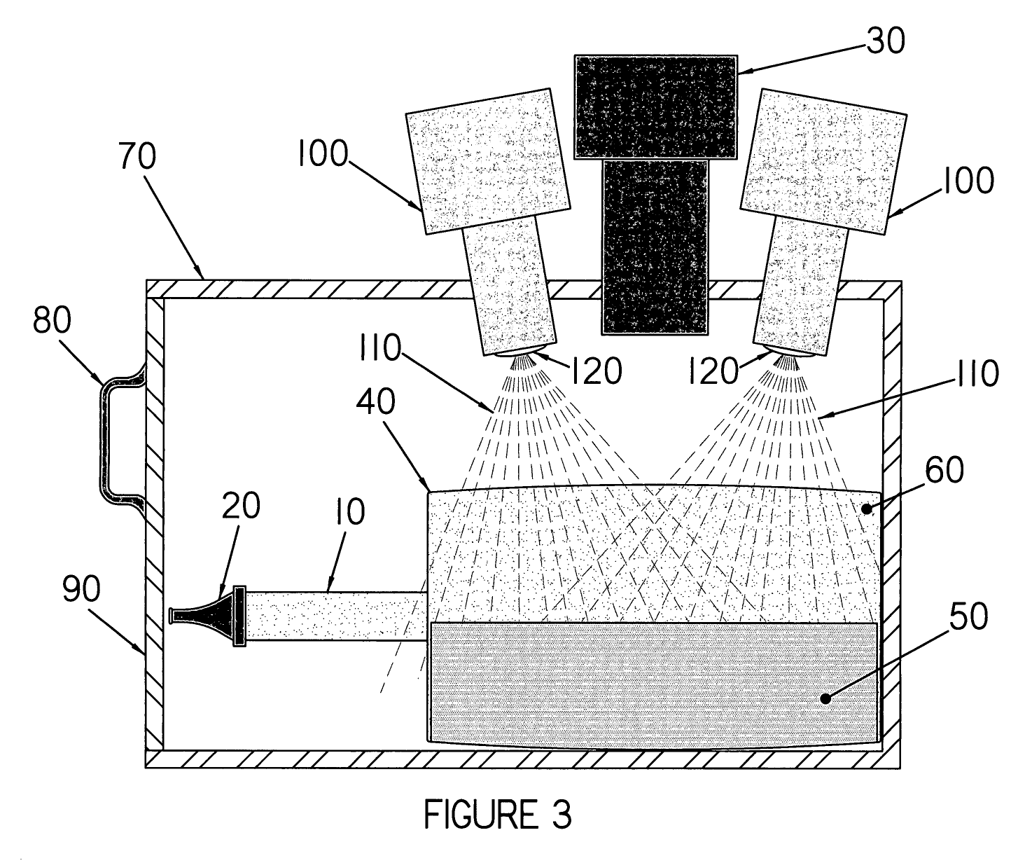

FIG. 3 shows a 2-dimensional side view of air sample bag test chamber. The chamber consists of a rigid, light-proof enclosure 70 with a door 90 to allow for a fully inflated sample bag to be placed inside 40. The door 90 can be opened by pulling on the handle 80. When the fully inflated sample bag 40 is filled with the air sample 60, the uniform layer of MIP's 50 is arrayed in a non-overlapping manor to allow for the individual molecules of air 60 to interact with as much surface area of MIP coated surface 50 as possible. When the door is closed 90, all external light is blocked, and the internal high intensity suitable wavelength light sources are activated 100. When activated, they produce an emission of suitable wavelength light 110 that is diffused through a small lens 120 to allow for a more uniform coverage area. Upon interaction of the suitable wavelength light 110 with the molecular concentration brought about by the MIP's 50, the molecules could now fluoresce. Since each type of MIP 50 that is coated inside the air sample bag 40 will target a specific molecular species, the pattern of fluorescence will indicate what species are present in the air sample 60. The resulting illumination brought about by fluorescence due to interaction of the suitable wavelength light 110 and the molecular species can be examined by a suitable, highly sensitive light-sensing device 30 such as a photomultiplier tube or CCD camera. If discrete rows or columns of specially targeted MIP's are arranged inside the air sample bag, then a chemical “fingerprint” of the sample can be made, and compared with a database of known chemical “fingerprints”. A small computer connected to the sensitive light sensing device 30 could utilize image processing techniques to further enhance the information to allow for more accurate and precise testing.

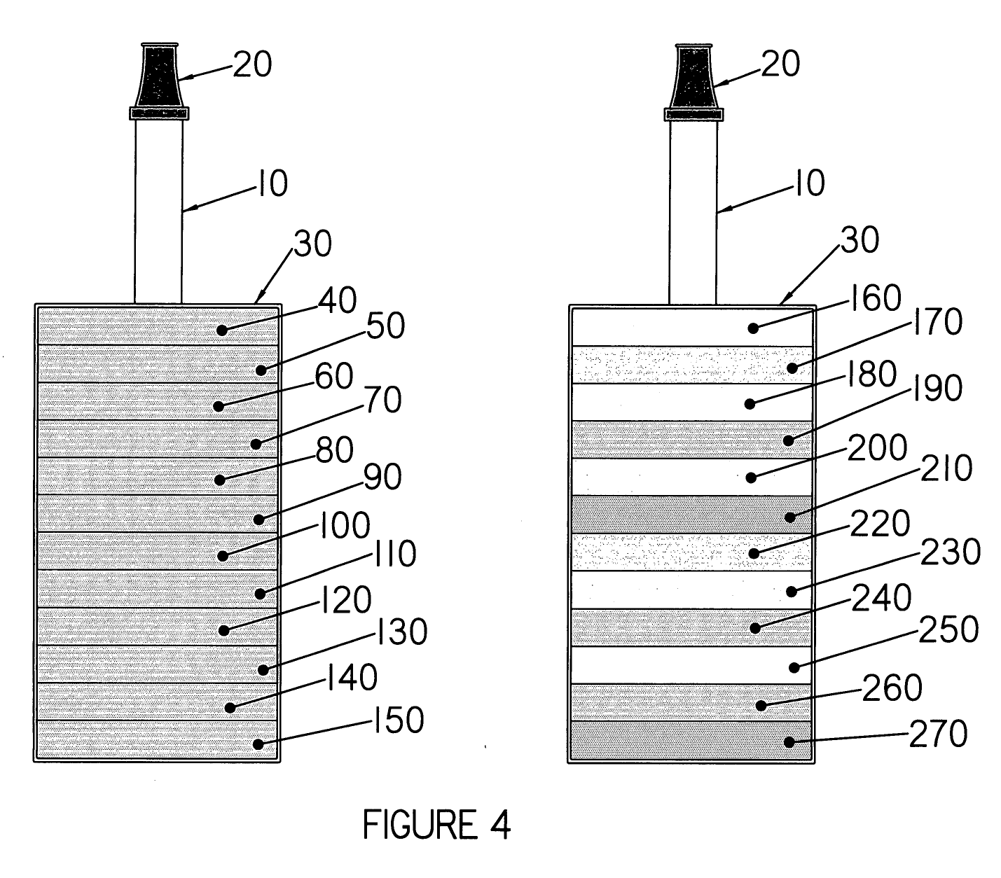

FIG. 4 shows a 2-dimensional top view of an uninflated, flexible plastic disposable air sample bag 30. To inflate the inflatable air sample bag, one needs to blow air into it. This is accomplished by placing ones lips onto the rigid, plastic nozzle 20 that is designed in such a way as to allow air into the flexible air sample bag, but prevent it from escaping. The rigid, plastic nozzle 20 connects to the main body of the flexible air sample bag 30 by a small flexible plastic tube 10 which will channel all the air inside the flexible air sample bag 30. The inside of the flexible air sample bag 30 is coated with discrete rows of specifically targeted MIP's; each designed to target a different molecular specie. One row of specifically designed MIP's could be used for a control 40 to indicate that the suitable wavelength light source and sensitive light sensor are working properly. Consecutive discrete rows of MIP's could be designed to detect, or bond to specific molecules such as Nitric Oxide 50 and Carbon Monoxide 60 to name just a few. As more distinct chemical precursors are identified by medical research identifying specific cancers or ailments, each could be added to the combination of discrete rows of MIP's. Each discrete row of MIP's will not be identified in this patent, due to the fact that medical research has not identified that many of them yet. As each chemical precursor becomes known, a specific MIP will be designed to target or sequester that specific molecular specie. Each new chemical specie will have its own distinct MIP row to be used for detection. If the specific chemical compound is contained in the air sample, then the appropriate MIP row will fluoresce under suitable wavelength illumination. In the preferred embodiment of the invention, an attached computer will be able to identify (by user input or a bar code on the air sample bag) what types of MIP's are contained inside the air sample bag. This will allow the computer to make intelligent, accurate and precise measurements as to what the air sample contains. As the air sample bag 30 is filled with an air sample, placed inside the test chamber and illuminated with suitable wavelength light, the discrete rows of specifically targeted MIP's will glow if they have sequestered enough material for detection. When illuminated with suitable wavelength light, each air sample bag will have a row of “control” MIP's 160 that will glow, or fluoresce strongly to indicate that both the suitable wavelength light source and the sensitive light sensor are working properly. If a breath sample contained a high amount of Nitric Oxide for example, the discrete row of MIP's specifically designed to target Nitric Oxide 50 will glow brightly under suitable wavelength light 170. This will tell the user that the breath sample just tested contains Nitric Oxide, and based on the intensity of light brought about by fluorescence, a determination as to the amount of Nitric Oxide can be made. If a discrete row of MIP's that are specifically designed to target Ammonia 150 is placed inside the air sample bag and illuminated with suitable wavelength light, and no fluorescence is indicated 270, then it can be determined that no ammonia, or trace amounts too low to be detected are in the air sample. As more and more medical research is done on chemical precursors, and more become known, specific MIP's could be designed to target these chemical precursors. Although twelve discrete rows of different MIP's are shown in the patent, there is no reason to limit that number to twelve. Instead of discreet rows of different MIP's, it could just as easily be a checkerboard matrix of different MIP's could be realized to give enhance analysis ability.

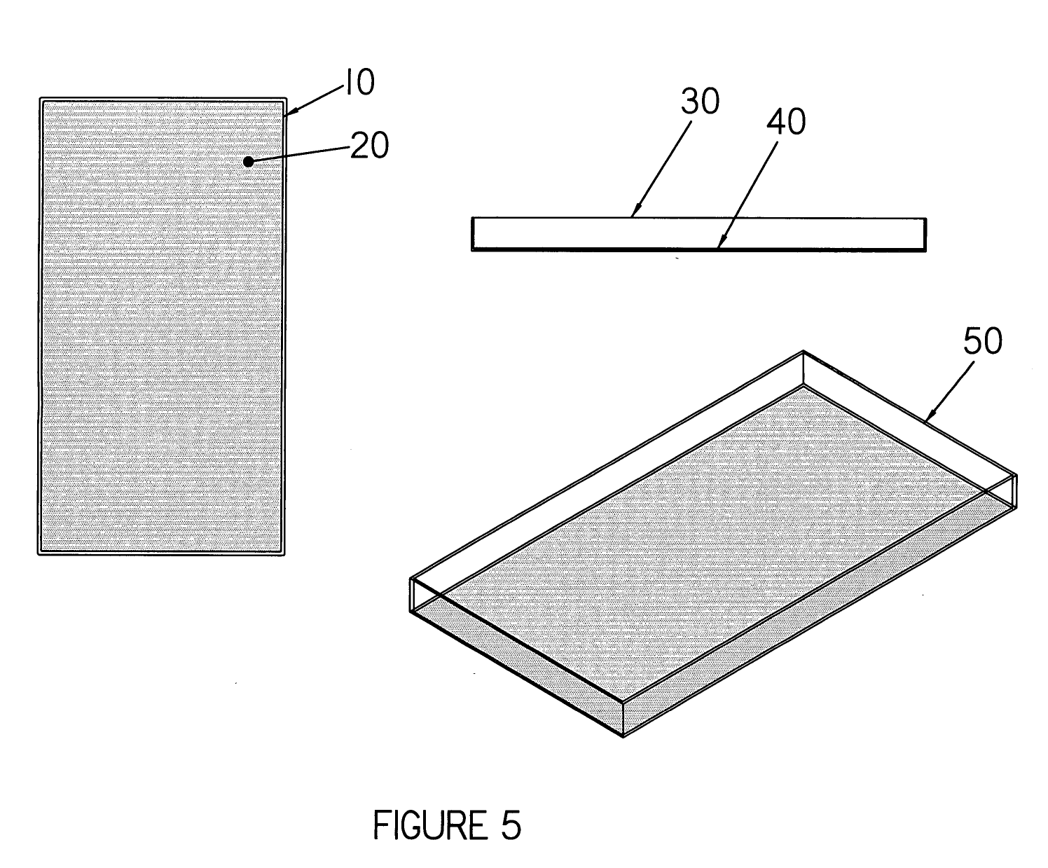

FIG. 5 shows several views of a rigid plastic liquid sample tray. The top view of the liquid sample tray 10 contains a coating of MIP's 20 that will be used to detect the presence or various chemical species. A side view of the liquid sample tray 30 indicates how the uniform layer of MIP's is coated on the inside surface 40 of the liquid sample tray. A three dimensional view of the liquid sample tray is shown 50 to give an indication as to the construction.

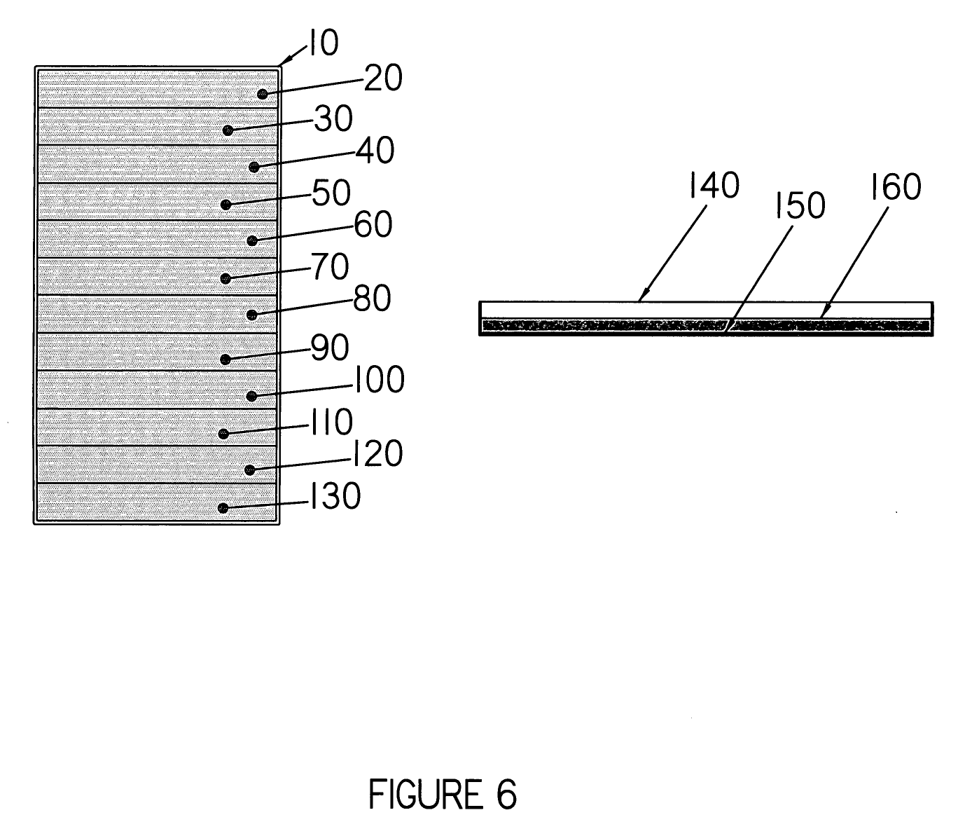

FIG. 6 shows a 2-dimensional view of a disposable, rigid liquid sample tray 10. The inside surface of the disposable rigid liquid sample tray 10 is coated with discrete rows of specifically targeted MIP's; each designed to target a different molecular specie. One row of specifically designed MIP's could be used for a control 20 to indicate that the suitable wavelength light source and sensitive light sensor are working properly. Consecutive discrete rows of MIP's could be designed to detect, or bond to specific molecules such as Ammonia 30 and THC 40 (if one wished to do drug testing) to name just a few. As more distinct chemical precursors are identified by medical research identifying specific cancers, ailments or drug testing, each could be added to the combination of discrete rows of MIP's. Each discrete row of MIP's will not be identified in this patent, due to the fact that medical research has not identified that many of them yet. As each chemical precursor becomes known, a specific MIP will be designed to target or sequester that specific molecular specie. Each new chemical specie will have its own distinct MIP row to be used for detection. If the specific chemical compound is contained in the air sample, then the appropriate MIP row will fluoresce under suitable wavelength illumination. In the preferred embodiment of the invention, an attached computer will be able to identify (by user input or a bar code on the air sample bag) what types of MIP's are contained inside the air sample bag. This will allow the computer to make intelligent, accurate and precise measurements as to what the air sample contains. As the liquid 160 is poured into the liquid sample container 140 and comes into direct contact with the MIP's 150, an interaction occurs between the specific chemical compounds contained in the liquid sample and the specially designed MIP's. When the filled liquid sample tray is placed inside the test chamber and illuminated with suitable wavelength light, the discrete rows of specifically targeted MIP's will glow if they have sequestered enough material for detection. When illuminated with suitable wavelength light, each liquid sample tray will have a row of “control” MIP's that will glow, or fluoresce strongly to indicate that both the suitable wavelength light source and the sensitive light sensor are working properly.

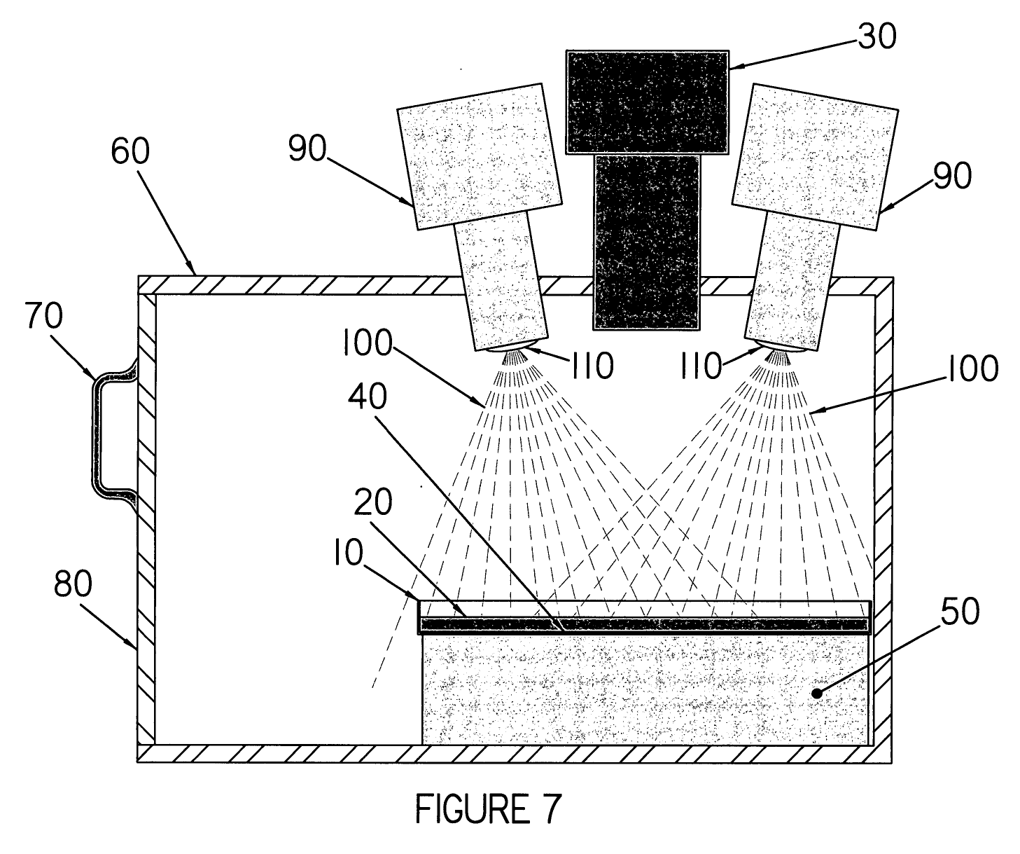

FIG. 7 shows a 2-dimensional side view of liquid sample tray test chamber (identical to that of the previously described air sample chamber). The chamber consists of a rigid, light-proof enclosure 60 with a door 80 to allow for a filled liquid sample tray to be placed inside 10. The door 80 can be opened by pulling on the handle 70. When the liquid sample tray 10 is filled with the liquid sample 20, the uniform layer of MIP's 40 will make direct physical contact with the liquid. When the door is closed 80, all external light is blocked, and the internal high intensity suitable wavelength light sources are activated 90. When activated, they produce an emission of suitable wavelength light 100 that is diffused through a small lens 110 to allow for a more uniform coverage area. Upon interaction of the suitable wavelength light 100 with the molecular concentration brought about by the MIP's 40, the molecules could now fluoresce. Since each type of MIP 40 that is coated inside the liquid sample tray 10 will target a specific molecular species, the pattern of fluorescence will indicate what species are present in the liquid sample 20. The resulting illumination brought about by fluorescence due to interaction of the suitable wavelength light 100 and the molecular species can be examined by a suitable, highly sensitive light-sensing device 30 such as a photomultiplier tube or CCD camera. If discrete rows or columns of specially targeted MIP's are arranged inside the liquid sample tray, then a chemical “fingerprint” of the sample can be made, and compared with a database of known chemical “fingerprints”. A small computer connected to the sensitive light sensing device 30 could utilize image processing techniques to further enhance the information to allow for more accurate and precise testing. A small plastic or wooden spacer block 50 would be used to allow for the height of the liquid sample tray to be fully illuminated and brought into the focal length.

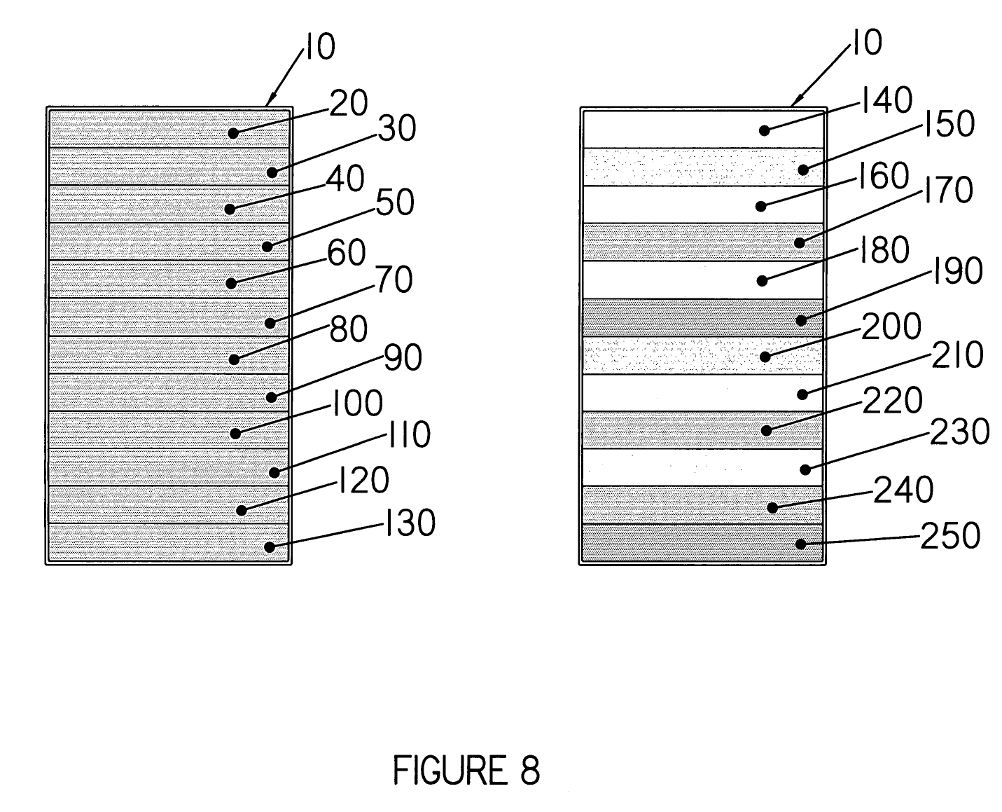

FIG. 8 shows a 2-dimensional top view of a liquid sample tray 10. The inside of the liquid sample tray 10 is coated with discrete rows of specifically targeted MIP's; each designed to target a different molecular specie. One row of specifically designed MIP's could be used for a control 20 to indicate that the suitable wavelength light source and sensitive light sensor are working properly. Consecutive discrete rows of MIP's could be designed to detect, or bond to specific molecules such as Ammonia 30 and THC 130 to name just a few. As more distinct chemical precursors are identified by medical research identifying specific cancers, ailments or drug testing, each could be added to the combination of discrete rows of MIP's. Each discrete row of MIP's will not be identified in this patent, due to the fact that medical research has not identified that many of them yet. As each chemical precursor becomes known, a specific MIP will be designed to target or sequester that specific molecular specie. Each new chemical specie will have its own distinct MIP row to be used for detection. If the specific chemical compound is contained in the liquid sample, then the appropriate MIP row will fluoresce under suitable wavelength illumination. In the preferred embodiment of the invention, an attached computer will be able to identify (by user input or a bar code on the air sample bag) what types of MIP's are contained inside the air sample bag. One type of MIP coated tray for the detection of cancer, one for drug testing, one for pregnancy determination, etc., each identified by a coded bar code to correlate information to the computer during analysis. This will allow the computer to make intelligent, accurate and precise measurements as to what the liquid sample contains. As the liquid sample tray 10 is filled with liquid (urine, water, blood, etc.) and placed inside the test chamber and illuminated with suitable wavelength light, the discrete rows of specifically targeted MIP's will glow if they have sequestered enough material for detection. When illuminated with suitable wavelength light, each liquid sample tray will have a row of “control” MIP's 20 that will glow, or fluoresce strongly to indicate that both the suitable wavelength light source and the sensitive light sensor are working properly. If a liquid sample tray contained liquid with a high amount of Ammonia for example, the discrete row of MIP's specifically designed to target Ammonia 30 will glow brightly under suitable wavelength light 150. This will tell the user that the liquid sample just tested contains ammonia, and based on the intensity of light brought about by fluorescence, a determination as to the amount of ammonia can be made. If a discrete row of MIP's that are specifically designed to target THC 130 is contained inside the liquid sample tray and illuminated with suitable wavelength light, and no fluorescence is indicated 250, then it can be determined that no THC, or only trace amounts too low to be detected are in the liquid sample. As more and more medical research is done on chemical precursors and biomarkers, and more become known, specific MIP's could be designed to target these chemical precursors. Although twelve discrete rows of different MIP's are shown in the patent, there is no reason to limit that number to twelve. Instead of discreet rows of different MIP's, it could just as easily be a checkerboard matrix of different MIP's could be realized to give enhance analysis ability.

In addition to simply looking at the overall illumination due to fluorescence, it is also beneficial to look at a lifetime-gated response of the sample. Lifetime gating refers to the ability of rapid extinction of a suitable wavelength illumination source while a sample is fluorescing, and measuring the time delay for the glow to cease. This time could be anywhere from microseconds to minutes. The differences in the amount of time before the glow is no longer detectable gives additional information as to the nature of the sample. To be technically correct, fluorescence is defined as the ability of a substance to emit light of a wavelength that is shifted from the illumination source while it is illuminated, while phosphorescence is defined as the ability to emit light after extinction of the illumination source. Based upon that definition, the described invention will look at the fluorescence and phosphorescence of a sample to give more precise and accurate results. A database of known phosphorescence lifetimes could be compared to the sample to make a more accurate determination of the composition of the unknown sample.

### REFERENCE NUMERALS

FIG. 1:
10 Flexible plastic inlet tube for providing path of breath sample to the bag.
20 One way, hard plastic valve to allow one way airflow to the bag.
30 Single inside surface of plastic inflatable bag that has been coated with various types of MIP's.
40 Main body of uninflated, flexible plastic inflatable bag that will store the air sample.
50 Side view of the one way, hard plastic valve to allow one way airflow to the bag.
60 Side view of flexible plastic inlet tube for providing path of breath sample to the bag.
70 Side view of main body of empty, flexible, plastic inflatable bag that will store the air sample.
80 Side view of single inside surface of plastic inflatable bag that has been coated with various types of MIP's.

FIG. 2:
10 Flexible plastic inlet tube for providing path of breath sample to the bag.
20 One way, hard plastic valve to allow one way airflow to the bag.
30 Single inside surface of plastic inflatable bag that has been coated with various types of MIP's.
40 Main body of uninflated, flexible plastic inflatable bag that will store the air sample.
50 Subject or patient that is having a non-invasive breath analysis performed.
60 Three-Dimensional view of flexible sample bag in the process of inflation.
70 Three-Dimensional view of fully inflated flexible sample bag.

FIG. 3:
10 Flexible plastic inlet tube for providing path of breath sample to the bag.
20 Side view of one way, hard plastic valve to allow one way airflow to the bag.
30 Sensitive light sensor such as a photomultiplier tube or a CCD camera.
40 Sid view of main body of flexible, plastic inflatable bag that has been fully inflated with the air sample.
50 Side view of MIP coated surface of the inside of the transparent flexible air sample bag.
60 Side view of fully inflated sample bag showing a schematic representation indicating gas molecules.
70 Side view of light proof test chamber that will house the sample bag.
80 Handle to open door to allow access of the air sample bag.
90 Door to allow access of the air sample bag.
100 Specific wavelength high-intensity light source used to illuminate the MIP's inside the flexible air sample bag.
110 Light rays of specific wavelength high-intensity light that are used to illuminate the MIP's inside the flexible air sample bag.
120 Lens that will spread out the light rays of to provide a wide, uniform illumination.

FIG. 4:
10 Flexible plastic inlet tube for providing path of breath sample to the bag.
20 One way, hard plastic valve to allow one way airflow to the bag.
30 Main body of flexible, plastic inflatable bag that will store the air sample.
40 Discrete strip of MIP's located inside the plastic inflatable bag that has been coated with a very specific type of MIP that will be used as a control.
50 Discrete strip of MIP's located inside the plastic inflatable bag that has been coated with a very specific type of MIP that will be used to detect the presence of a specific molecule.
60 Discrete strip of MIP's located inside the plastic inflatable bag that has been coated with a very specific type of MIP that will be used to detect the presence of a specific molecule.
70 Discrete strip of MIP's located inside the plastic inflatable bag that has been coated with a very specific type of MIP that will be used to detect the presence of a specific molecule.
80 Discrete strip of MIP's located inside the plastic inflatable bag that has been coated with a very specific type of MIP that will be used to detect the presence of a specific molecule.
90 Discrete strip of MIP's located inside the plastic inflatable bag that has been coated with a very specific type of MIP that will be used to detect the presence of a specific molecule.
100 Discrete strip of MIP's located inside the plastic inflatable bag that has been coated with a very specific type of MIP that will be used to detect the presence of a specific molecule.
110 Discrete strip of MIP's located inside the plastic inflatable bag that has been coated with a very specific type of MIP that will be used to detect the presence of a specific molecule.
120 Discrete strip of MIP's located inside the plastic inflatable bag that has been coated with a very specific type of MIP that will be used to detect the presence of a specific molecule.
130 Discrete strip of MIP's located inside the plastic inflatable bag that has been coated with a very specific type of MIP that will be used to detect the presence of a specific molecule.
140 Discrete strip of MIP's located inside the plastic inflatable bag that has been coated with a very specific type of MIP that will be used to detect the presence of a specific molecule.
150 Discrete strip of MIP's located inside the plastic inflatable bag that has been coated with a very specific type of MIP that will be used to detect the presence of a specific molecule.
160 Discrete strip of MIP's located inside the plastic inflatable bag that has been coated with a very specific type of MIP that will be used as a control, shown strongly fluorescing due to illumination of suitable wavelength light.
170 Discrete strip of MIP's located inside the plastic inflatable bag that has been coated with a very specific type of MIP that will be used to detect the presence of a specific molecule shown moderately fluorescing due to presence of target specie.
180 Discrete strip of MIP's located inside the plastic inflatable bag that has been coated with a very specific type of MIP that will be used to detect the presence of a specific molecule shown strongly fluorescing due to presence of target specie.
190 Discrete strip of MIP's located inside the plastic inflatable bag that has been coated with a very specific type of MIP that will be used to detect the presence of a specific molecule shown weakly fluorescing due to presence of target specie.
200 Discrete strip of MIP's located inside the plastic inflatable bag that has been coated with a very specific type of MIP that will be used to detect the presence of a specific molecule shown strongly fluorescing due to presence of target specie.
210 Discrete strip of MIP's located inside the plastic inflatable bag that has been coated with a very specific type of MIP that will be used to detect the presence of a specific molecule shown without fluorescing, indicating absence of target specie.
220 Discrete strip of MIP's located inside the plastic inflatable bag that has been coated with a very specific type of MIP that will be used to detect the presence of a specific molecule shown moderately fluorescing due to presence of target specie.
230 Discrete strip of MIP's located inside the plastic inflatable bag that has been coated with a very specific type of MIP that will be used to detect the presence of a specific molecule shown strongly fluorescing due to presence of target specie.
240 Discrete strip of MIP's located inside the plastic inflatable bag that has been coated with a very specific type of MIP that will be used to detect the presence of a specific molecule shown weakly fluorescing due to presence of target specie.
250 Discrete strip of MIP's located inside the plastic inflatable bag that has been coated with a very specific type of MIP that will be used to detect the presence of a specific molecule shown strongly fluorescing due to presence of target specie.
260 Discrete strip of MIP's located inside the plastic inflatable bag that has been coated with a very specific type of MIP that will be used to detect the presence of a specific molecule shown weakly fluorescing due to presence of target specie.
270 Discrete strip of MIP's located inside the plastic inflatable bag that has been coated with a very specific type of MIP that will be used to detect the presence of a specific molecule shown without fluorescing, indicating absence of target specie.

FIG. 5:
10 Rigid, plastic tray used for housing liquid samples.
20 Uniform coating of MIP's covering inside surface.
30 Side view of rigid liquid sample tray.
40 Side view of uniform coating of MIP's covering inside surface.
50 Three-dimensional view of liquid sample tray with uniform coating of MIP's.

FIG. 6:
10 Rigid, plastic tray used for housing liquid samples.
20 Discrete strip of MIP's located inside the rigid, plastic liquid sample tray that has been coated with a very specific type of MIP that will be used as a control.
30 Discrete strip of MIP's located inside the rigid, plastic liquid sample tray that has been coated with a very specific type of MIP that will be used to detect the presence of a specific molecule.
40 Discrete strip of MIP's located inside the rigid, plastic liquid sample tray that has been coated with a very specific type of MIP that will be used to detect the presence of a specific molecule.
50 Discrete strip of MIP's located inside the rigid, plastic liquid sample tray that has been coated with a very specific type of MIP that will be used to detect the presence of a specific molecule.
60 Discrete strip of MIP's located inside the rigid, plastic liquid sample tray that has been coated with a very specific type of MIP that will be used to detect the presence of a specific molecule.
70 Discrete strip of MIP's located inside the rigid, plastic liquid sample tray that has been coated with a very specific type of MIP that will be used to detect the presence of a specific molecule.
80 Discrete strip of MIP's located inside the rigid, plastic liquid sample tray that has been coated with a very specific type of MIP that will be used to detect the presence of a specific molecule.
90 Discrete strip of MIP's located inside the rigid, plastic liquid sample tray that has been coated with a very specific type of MIP that will be used to detect the presence of a specific molecule.
100 Discrete strip of MIP's located inside the rigid, plastic liquid sample tray that has been coated with a very specific type of MIP that will be used to detect the presence of a specific molecule.
110 Discrete strip of MIP's located inside the rigid, plastic liquid sample tray that has been coated with a very specific type of MIP that will be used to detect the presence of a specific molecule.
120 Discrete strip of MIP's located inside the rigid, plastic liquid sample tray that has been coated with a very specific type of MIP that will be used to detect the presence of a specific molecule.
130 Discrete strip of MIP's located inside the rigid, plastic liquid sample tray that has been coated with a very specific type of MIP that will be used to detect the presence of a specific molecule.
140 Side view of Rigid, plastic tray used for housing liquid samples.
150 Side view of discrete strips of MIP's located inside the rigid, plastic liquid sample tray.
160 Side view of liquid placed inside the rigid, plastic liquid sample tray.

FIG. 7:
10 Side view of rigid, plastic liquid sample tray.
20 Side view of rigid, plastic liquid sample tray partially filled with test liquid
30 Sensitive light sensor such as a photomultiplier tube or a CCD camera.
40 Side view of discrete strips of MIP's located inside the rigid, plastic liquid sample tray.
50 Plastic or wooden block used to raise the rigid plastic liquid sample tray to the focal point of the sensitive light sensor, and allow for complete overall illumination of the sample by the suitable wavelength light source.
60 Side view of light proof test chamber that will house the liquid sample tray.
70 Handle to open door to allow access of the liquid sample tray.
80 Door to allow access of the liquid sample tray.
90 Specific wavelength high-intensity light source used to illuminate the MIP's inside the liquid sample tray.
100 Light rays of specific wavelength high-intensity light that are used to illuminate the MIP's inside the liquid sample tray.
110 Lens that will spread out the light rays of to provide a wide, uniform illumination.

FIG. 8:
10 Main body of rigid, plastic, disposable liquid sample tray.
20 Discrete strip of MIP's located inside the rigid, plastic, disposable liquid sample tray that has been coated with a very specific type of MIP that will be used as a control.
30 Discrete strip of MIP's located inside the rigid, plastic, disposable liquid sample tray that has been coated with a very specific type of MIP that will be used to detect the presence of a specific molecule.
40 Discrete strip of MIP's located inside the rigid, plastic, disposable liquid sample tray that has been coated with a very specific type of MIP that will be used to detect the presence of a specific molecule.
50 Discrete strip of MIP's located inside the rigid, plastic, disposable liquid sample tray that has been coated with a very specific type of MIP that will be used to detect the presence of a specific molecule.
60 Discrete strip of MIP's located inside the rigid, plastic, disposable liquid sample tray that has been coated with a very specific type of MIP that will be used to detect the presence of a specific molecule.
70 Discrete strip of MIP's located inside the rigid, plastic, disposable liquid sample tray that has been coated with a very specific type of MIP that will be used to detect the presence of a specific molecule.
80 Discrete strip of MIP's located inside the rigid, plastic, disposable liquid sample tray that has been coated with a very specific type of MIP that will be used to detect the presence of a specific molecule.
90 Discrete strip of MIP's located inside the rigid, plastic, disposable liquid sample tray that has been coated with a very specific type of MIP that will be used to detect the presence of a specific molecule.
100 Discrete strip of MIP's located inside the rigid, plastic, disposable liquid sample tray that has been coated with a very specific type of MIP that will be used to detect the presence of a specific molecule.
110 Discrete strip of MIP's located inside the rigid, plastic, disposable liquid sample tray that has been coated with a very specific type of MIP that will be used to detect the presence of a specific molecule.
120 Discrete strip of MIP's located inside the rigid, plastic, disposable liquid sample tray that has been coated with a very specific type of MIP that will be used to detect the presence of a specific molecule.
130 Discrete strip of MIP's located inside the rigid, plastic, disposable liquid sample tray that has been coated with a very specific type of MIP that will be used to detect the presence of a specific molecule.
140 Discrete strip of MIP's located inside the rigid, plastic, disposable liquid sample tray that has been coated with a very specific type of MIP that will be used as a control shown strongly fluorescing due to illumination of suitable wavelength light.
150 Discrete strip of MIP's located inside the rigid, plastic, disposable liquid sample tray that has been coated with a very specific type of MIP that will be used to detect the presence of a specific molecule shown moderately fluorescing due to presence of target specie.
160 Discrete strip of MIP's located inside the rigid, plastic, disposable liquid sample tray that has been coated with a very specific type of MIP that will be used to detect the presence of a specific molecule shown strongly fluorescing due to presence of target specie.
170 Discrete strip of MIP's located inside the rigid, plastic, disposable liquid sample tray that has been coated with a very specific type of MIP that will be used to detect the presence of a specific molecule shown weakly fluorescing due to presence of target specie.
180 Discrete strip of MIP's located inside the rigid, plastic, disposable liquid sample tray that has been coated with a very specific type of MIP that will be used to detect the presence of a specific molecule shown strongly fluorescing due to presence of target specie.
190 Discrete strip of MIP's located inside the rigid, plastic, disposable liquid sample tray that has been coated with a very specific type of MIP that will be used to detect the presence of a specific molecule shown without fluorescing, indicating absence of target specie.
200 Discrete strip of MIP's located inside the rigid, plastic, disposable liquid sample tray that has been coated with a very specific type of MIP that will be used to detect the presence of a specific molecule shown moderately fluorescing due to presence of target specie.
210 Discrete strip of MIP's located inside the rigid, plastic, disposable liquid sample tray that has been coated with a very specific type of MIP that will be used to detect the presence of a specific molecule shown strongly fluorescing due to presence of target specie.
220 Discrete strip of MIP's located inside the rigid, plastic, disposable liquid sample tray that has been coated with a very specific type of MIP that will be used to detect the presence of a specific molecule shown weakly fluorescing due to presence of target specie.
230 Discrete strip of MIP's located inside the rigid, plastic, disposable liquid sample tray that has been coated with a very specific type of MIP that will be used to detect the presence of a specific molecule shown strongly fluorescing due to presence of target specie.
240 Discrete strip of MIP's located inside the rigid, plastic, disposable liquid sample tray that has been coated with a very specific type of MIP that will be used to detect the presence of a specific molecule shown weakly fluorescing due to presence of target specie.
250 Discrete strip of MIP's located inside the rigid, plastic, disposable liquid sample tray that has been coated with a very specific type of MIP that will be used to detect the presence of a specific molecule shown without fluorescing, indicating absence of target specie.

We claim the following:

1. a method of utilizing molecularly imprinted polymers to enable sequestering and concentrating of specific chemical compounds indicative of diseases and ailments

2. a method of illuminating molecularly imprinted polymers with specific narrow band ultraviolet sources to cause fluorescence

3. a method of analyzing exhaled breath utilizing the process in claim 1 and claim 2

4. a method of analyzing bodily fluids utilizing the process in claim 1 and claim 2
